(19) United States
(12) Patent Application Publication
(10) Pub. No.: US 2018/0140375 A1
(43) Pub. Date: May 24, 2018

(54) VIDEOSCOPIC SURGERY AND ENDOSCOPIC SURGERY PROCEDURES EMPLOYING OSCILLATING IMAGES

(71) Applicant: Roger de la Torre, Columbia, MO (US)

(72) Inventor: Roger de la Torre, Columbia, MO (US)

(21) Appl. No.: 15/821,481

(22) Filed: Nov. 22, 2017

Related U.S. Application Data
(60) Provisional application No. 62/425,754, filed on Nov. 23, 2016.

(51) Int. Cl. A61B 90/00; A61B 1/06; A61B 34/37; A61B 1/00; A61B 1/04; A61B 1/313; A61M 5/00; A61K 49/00

(52) U.S. Cl. CPC A61B 90/361; A61B 1/0638; A61B 34/37; A61B 1/0005; A61B 1/043; A61B 2090/304; A61B 1/313; A61M 5/007; A61K 49/0034; A61B 2090/373; A61B 1/00009

(57) ABSTRACT

This disclosure is directed to improving the overall image of the patient's tissues displayed on the television monitor or optical googles or glasses and viewed by the surgeon or endoscopist when using dyes or other processes such as ICG.

This patent application claims the benefit of the filing date of provisional patent application Ser. No. 62/425,754, which was filed on Nov. 23, 2017.

### BACKGROUND

Videoscopic surgery is a specialized way of performing surgical procedures with the assistance of a videocamera that can be connected to a monitor or to optic goggles or glasses. Although this can be used in more conventional, open surgery, commonly this is used in minimally invasive surgery, such as laparoscopic, thorascopic or arthroscopic surgery, as well as for endoscopy and endoscopic procedures. Typically, during minimally invasive surgery or robotic surgery, one or more small incision(s) are made into the patient and a tubular trocar is then inserted through the patient's tissues. A videoscope is introduced through one of the trocars and into the patient's tissues. The videoscope is usually either a rigid or a flexible tube with an attached camera and light source. Similarly, in endoscopy, an endoscope is inserted into a patient's natural orifice for diagnostic purposes or to perform any of a variety of procedures. Here too, the endoscope has an attached light source and camera.

The camera transmits an image of the patient's tissues onto a television monitor or onto glasses or goggles. The surgeon uses the projected image of the tissues to diagnose or perform a procedure using instruments usually inserted through the other trocars or through channels in the endoscope.

The transmitted image of the patient's tissues can be altered or enhanced. This is often accomplished with the use of intravenous chemicals or dyes such as fluorescein dye or indocyanine green dye (ICG) injected into the patient, and by changing the wavelength of light or by the use of optic filters as well as other processes. For example, ICG injected into the patient is attracted to proteins in the patient's body. Certain wavelengths of light will excite the fluorescence of ICG following the intravenous injection. This can then be recorded with a video camera that with certain optical filters will collect only the ICG induced fluorescence. This will initially display arterial, capillary and venous angiographic images on the television monitor viewed by the surgeon. Later, bile and the flow of bile through ductal structures will come into view. In a variety of videoscopic surgical and endoscopic procedures, the use of ICG and various wavelengths of light and optical filters enhance the visualization of anatomical structures and physiological functions.

In a variety of videoscopicsurgical and endoscopic procedures, the use of chemicals or dyes such as ICG when excited by wavelengths of light and viewed by optical filters enhance the visualization of anatomical structures and physiological functions. This will allow surgeons and endoscopists to view blood flow to tissues, bilious flow, and perhaps cancerous tissue during a procedure.

However, current methods of using such chemicals or dyes such as ICG to enhance visualization do not allow for real time visualization of the enhancements while also enabling the surgeon to visualize the patient's tissues in its native or natural appearance. Visualization of the enhancements displayed on the television monitor prevents normal color perception.

### BRIEF DESCRIPTION OF THE DRAWINGS

FIG. 1 is a representation of the apparatus and method of this disclosure used to enhance visualization in a videoscopic surgical procedure.

### DESCRIPTION

This disclosure is directed to improving the overall image of the patient's tissues displayed on the television monitor or optical googles or glasses and viewed by the surgeon or endoscopist when using dyes or other processes such as ICG.

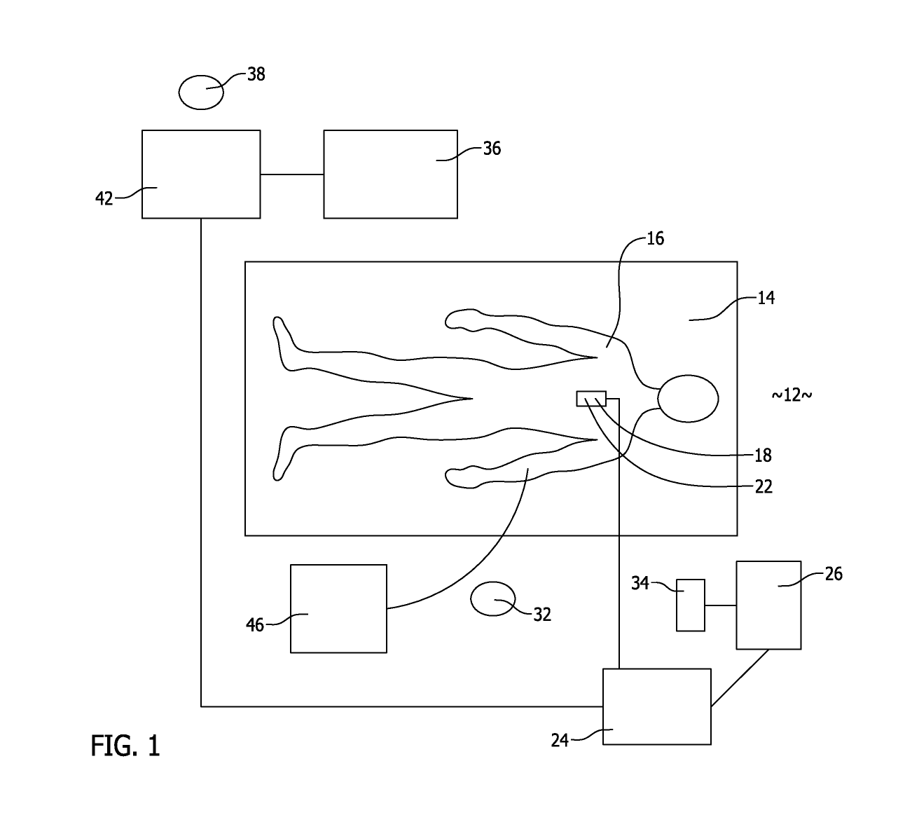

FIG. 1 is a representation of one operative environment of the apparatus of this disclosure that enhances visualization in a videoscopic surgical procedure, and its method of operation. FIG. 1 is only one example of an operative environment for the apparatus and method. There could be many other equivalent types of environments.

In FIG. 1 an operating room 12 is represented. A surgical table 14 is inside the operating room 12. A patient 16 is represented on the surgical table 14. Although the apparatus and method of this disclosure can be used in more conventional, open surgery, a minimally invasive surgical procedure, such as laparoscopic, thorascopic or arthroscopic, as well as in endoscopy and endoscopic procedure is represented.

In the minimally invasive surgical procedure represented in FIG. 1, one or more small incision(s) are made into the patient and one or more tubular trocars 18 are then inserted through the patient's tissues. A videoscope 22 with a camera and light source represented schematically in FIG. 1 is introduced through one of the trocars 18 and into the patient's tissues.

Similarly, in endoscopy, an endoscope is inserted into a patient's natural orifice for diagnostic purposes or to perform any of a variety of procedures. Here to, the endoscope has an attached light source and camera.

The videoscope 22 is operatively communicated with computerized image processing equipment 24. The computerized image processing equipment 24 is operatively communicated with a video display 26. The video display 26 could be a television monitor, video glasses or video goggles. The light source of the videoscope illuminates the surgical site and the camera of the videoscope 22 detects images of a surgical site in the patient 16 and transmits the images to the computerized image processing equipment which in turn transmits signals to the video display 26. The video display 26 displays images of the surgical site detected by the camera to the surgeon 32 performing the surgical procedure. A manual control 34, such as a keyboard is operatively communicated with the video display 26 and can be used by the surgeon 32 to adjust the images displayed by the video display 26.

Alternatively, or in addition to the surgeon 32 performing the surgical procedure represented in FIG. 1, a robotic surgical system 36 could be employed. The robotic surgical system 36 is controlled by a surgeon 38 sitting at or standing next to the robotic control console 42. The computerized image processing equipment 24 also operatively communicates with the robotic control console 42. In the robotic control console 42, a video display of the image of the surgical site detected by the camera of the videoscope 22 is displayed to the surgeon 38.

Whether the minimally invasive surgical procedure is being performed by the surgeon 32 through the trocars 18 inserted through the small incisions made in the patient 16, or by the surgeon 38 operating the robotic surgical system 36, the camera of the videoscope 22 transmits images of the surgical site onto the video display 26 or onto the video display of the robotic control console 42. The surgeon uses the projected images of the tissues at the surgical site to diagnose or perform a procedure using instruments inserted through the trocar 18 or using the instruments of the robotic surgical system 36.

As stated earlier, the image of the patient's tissues at the surgical site transmitted to the video display 26 or the display of the robotic control console 42 can be altered or enhanced. This is often accomplished with the use of intravenous chemicals or dyes such as fluorescein dye or indocyanine green dye (ICG) injected into the patient, and by changing the wavelength of light provided by the videoscope 22 to the patient's interior, or by the use of optic fibers as well as other processes used to transmit changing wavelengths of light to the patient's tissues at the surgical site. This is represented by the dye injection supply and control 46 of FIG. 1. In the example of ICG, the ICG injected into the patient 16 is attracted to proteins in the patient's body. Certain wavelengths of light will excite the fluorescence of ICG following the intravenous injection. This can then be recorded with a video camera such as the camera of the videoscope 22, that with certain optical filters will collect only the ICG induced fluorescence. This will initially display on the video display 26, or on the display on the robotic control console 42, arterial, capillary and venous angiographic images that can be viewed by the surgeon 32, 38. Later, bile and the flow of bile through the ductal structures will come into view. In a variety of videoscopic surgical and endoscopic procedures, the use of chemicals or dyes such as ICG when excited by various wavelengths of light and viewed by optical filters enhance the visualization of anatomical structures and physiological functions at the surgical site. This allows surgeons and endoscopists to view blood flow to tissues, bilious flow, and perhaps cancerous tissue at the surgical site during a surgical procedure.

However, as stated earlier, current methods of using such chemicals or dyes such as ICG to enhance visualization do not allow for real time visualization of the enhancements while also enabling the surgeon to visualize the patient's tissue at the surgical site in its native or natural appearance. Visualization of the enhancements displayed on the video display 26 or on the display of the robotic control console 42 prevent normal color perception.

The above disadvantages are overcome by the apparatus of this disclosure and its method of use.

The computerized image processing equipment 24 of this disclosure is operable to oscillate images displayed on the video display 26 or the display of the robotic control console 42 between normal vision of the patient's tissue at the surgical site and images enhanced by wavelength(s) or optical fibers during an operative procedure. This enables the surgeon 32, 38 to view an overlay of the tissues at the surgical site that have been enhanced by ICG, while at the same time seeing the tissues at the surgical site as it would normally appear. By oscillating the images displayed on the video display 26 or the display of the robotic surgical system 36 between a normal image of the patient's tissues at the surgical site and enhanced images, such as after intravenous ICG infusion and excitation by a wavelength of light followed by optical fibers, the surgeon is able to view an overlay of the enhanced, altered image of the patient's tissues at the surgical site while also seeing the tissues as they would normally appear. Oscillating back and forth the image displayed between the enhanced image of the patient's tissues at the surgical site and the normal image of the patient's tissues at the surgical site enables the surgeon to concurrently see tissue that is altered and the same tissue as it normally appears. The frequency of the oscillating back and forth between the enhanced image and the normal image can be controlled by the surgeon 32 at the manual control 34 of the video display 26, or by the surgeon 38 at the robotic control console 42.

By oscillating images displayed on a monitor, goggles or glasses between a normal image of the patient's tissues and an enhanced image, such as after intravenous ICG infusion and excitation by a wavelength of light followed by optical filters, the surgeon is able to view an overlay of the enhanced, altered image of the tissues while also seeing tissue as it would normally appear. Oscillating back and forth the image displayed between the enhanced image of the patient's tissue and the normal image of the patient's tissue enables the surgeon to concurrently see tissue that is altered and the same tissue as it normally appears.

As various modifications could be made in the construction of the apparatus and its method of operation herein described and illustrated without departing from the scope of the invention, it is intended that all matter contained in the foregoing description or shown in the accompanying drawings shall be interpreted as illustrative rather than limiting. Thus, the breadth and scope of the present disclosure should not be limited by any of the above described exemplary embodiments, but should be defined only in accordance with the following claims appended hereto and their equivalents.

1. An apparatus that enhances visualization in a videoscopic surgical procedure, the apparatus comprising:
a videoscope that is operable to illuminate a surgical site and detect images of the surgical site;
computerized image processing equipment that is operatively communicated with the videoscope;
a display that is operatively communicated with the computerized image processing equipment; and,
the computerized image processing equipment being operable to control wavelengths of light transmitted by the videoscope and control oscillations between the wavelengths of light transmitted by the videoscope.

2. The apparatus of claim 1, further comprising:
the computerized image processing equipment being operable to control wavelengths of light transmitted by the videoscope between at least two different wavelengths of light and control oscillations between the at least two different wavelengths of light emitted by the videoscope.

3. The apparatus of claim 2, further comprising:
a supply of chemical that is operatively communicable with the surgical site, the chemical of the supply of chemical fluorescing when illuminated by one wavelength of light of the at least two wavelengths of light.

4. The apparatus of claim 3, further comprising:
the chemical is ICG.

5. The apparatus of claim 3, further comprising:
a chemical injection supply and control that is operable to control the supply of chemical that is communicated with the surgical site.

6. The apparatus of claim 2, further comprising:
the display is a video display monitor.

7. The apparatus of claim 2, further comprising:
a robotic surgical system;
a robotic control console operatively communicated with the robotic surgical system; and,
the computerized image processing equipment being operatively communicated with the robotic control console.

8. A apparatus of claim 7, further comprising:
the display being a display of the robotic control console.

9. An apparatus that enhances visualization in a videoscopic procedure, the apparatus comprising:
a camera that is operable to illuminate a surgical site and detect images of the surgical site;
a light source operatively connected to the camera and operable to illuminate the surgical site;
computerized image processing equipment operatively connected with the camera;
a video display operatively connected with the computerized image processing equipment and operable to display images of the surgical site detected by the camera; and,
the computerized image processing equipment being operable to oscillate images displayed on the video display between images illuminated by different wavelengths of light.

10. The apparatus of claim 9, further comprising:
the computerized image processing equipment being operable to control wavelengths of light transmitted by the light source between at least two different wavelengths of light and control oscillations between the at least two different wavelengths of light emitted by the light source.

11. The apparatus of claim 10, further comprising:
a supply of chemical that is operatively communicable with the surgical site, the chemical of the supply of chemical fluorescing when illuminated by one wavelength of light of the at least two wavelengths of light.

12. The apparatus of claim 11, further comprising:
the chemical is ICG.

13. The apparatus of claim 11, further comprising:
a chemical injection supply and control that is operable to control the supply of chemical that is communicated with the surgical site.

14. The apparatus of claim 10, further comprising:
the display is a video display monitor.

15. The apparatus of claim 10, further comprising:
a robotic surgical system;
a robotic control console operatively communicated with the robotic surgical system; and,
the computerized image processing equipment being operatively communicated with the robotic control console.

16. The apparatus of claim 15, further comprising:
the display being a display of the robotic control console.

17. A method of improving a displayed image on a television monitor viewed by a surgeon or endoscopist, the method comprising:
oscillating images of a surgical site displayed on a viewing monitor, goggles or glasses back and forth between an enhanced image such as after intravenous infusion of ICG and light excitation followed by optical filtration, and a normally displayed image.

18. The method of claim 17, further comprising:
the oscillating images being displayed on right and left eye displays of a three-dimensional monitor.

19. The method of claim 17, further comprising:
illuminating the surgical site with a first wavelength of light producing a first image of the oscillating images and a second wavelength of light producing a second image of the oscillating images.

20. The method of claim 17, further comprising:
oscillating images on a display of a robotic control console of a robotic surgical system.
